(19) United States
(12) Patent Application Publication
(10) Pub. No.: US 2025/0360021 A1
(43) Pub. Date: Nov. 27, 2025

(54) OSTOMY APPLIANCES

(71) Applicant: CONVATEC TECHNOLOGIES INC., Las Vegas, NV (US)

(72) Inventor: Monica Hill, Painsville, OH (US)

(21) Appl. No.: 19/295,656

(22) Filed: Aug. 10, 2025

Related U.S. Application Data
(63) Continuation of application No. PCT/US2024/014765, filed on Feb. 7, 2024.
(60) Provisional application No. 63/444,738, filed on Feb. 10, 2023.

(30) Foreign Application Priority Data
Mar. 14, 2023 (GB) 2303739.3

(51) Int. Cl. A61F 5/441; A61F 5/445

(52) U.S. Cl. CPC A61F 5/441; A61F 5/445

(57) ABSTRACT

A pouch having a vent aperture and a filter secured to the pouch with adhesive and covering the vent aperture. The filter includes an adhesive layer comprising the adhesive and an adsorbent layer on the adhesive layer. The filter also includes a gas permeable cover layer secured to the adhesive layer such that the adsorbent layer is covered by the gas permeable cover layer. The gas permeable cover layer has an outer profile and the adhesive layer has a corresponding outer profile which is in register with the outer profile of the gas permeable cover layer. The adsorbent layer is positioned on a first side of the adhesive layer so that a first portion of the first side of the adhesive layer is not covered by the adsorbent layer and the gas permeable cover layer is secured to the first portion of the first side of the adhesive layer.

### TECHNICAL FIELD OF THE INVENTION

The present invention relates to an ostomy appliance and a method of manufacturing such an appliance. The invention also relates to a filter for an ostomy appliance.

### BACKGROUND TO THE INVENTION

An ostomy is a surgical procedure which is performed when a patient has lost normal bowel or bladder function as a result of a disease, injury or other defect. Most ostomy procedures are performed on cancer patients. After the ostomy procedure has been performed, waste from the patient is expelled via a surgical opening called a “stoma” into an ostomy appliance such as an ostomy bag.

The waste includes gases and these gases can inflate the ostomy bag causing discomfort to the patient. Further, the gases can potentially overinflate the bag to the extent that the bag bursts, if left unvented. To counter the overinflation problem generally ostomy bags are provided with a vent aperture which allows the gases to leave the bag once they have passed through a filter. The filter removes the foul odour of the gases as they leave the ostomy bag.

A filter for an ostomy bag is described in EP 2 274 068 B1. EP 2 274 068 B1 notes that it is not possible to attach a filter to a ostomy bag with purely adhesive, and therefore a combination of heat welding and adhesive is provided to attach the filter to the bag. In particular, a cover layer of the filter is provided with a peripheral flange which is welded to the bag when the filter is attached to the ostomy bag.

A problem with the EP 2 274 068 B1 filter is that it requires a welding step to be attached to the bag, leading to the use of more energy during manufacture and a long manufacturing time. In addition, the peripheral flange increases the footprint of the filter and increases waste.

It is an object of the present invention to obviate or mitigate at least one problem with prior ostomy appliances and filters for use with such appliances, and/or to provide an improved ostomy appliance and/or filter.

### SUMMARY OF THE INVENTION

According to a broad aspect of the present invention, there is provided an ostomy appliance. The ostomy appliance may comprise a pouch. The pouch may have a vent aperture. The appliance may comprise a filter. The filter may be secured to the pouch with adhesive. The filter may cover the vent aperture. The filter may comprise an adhesive layer comprising the adhesive. The filter may comprise an adsorbent layer. The adsorbent layer may be arranged on the adhesive layer. The filter may comprise a gas permeable cover layer. The gas permeable cover layer may be secured to the adhesive layer such that the adsorbent layer is covered by the gas permeable cover layer. The gas permeable cover layer may have an outer profile. The adhesive layer may have a corresponding outer profile. The outer profile of the adhesive layer may be in register with the outer profile of the gas permeable cover layer. The adsorbent layer may be positioned on a first side of the adhesive layer so that a first portion of the first side of the adhesive layer is not covered by the adsorbent layer. The gas permeable cover layer may be secured to the first portion of the first side of the adhesive layer.

In one embodiment there is provided an ostomy appliance comprising:
a pouch having a vent aperture; and
a filter secured to the pouch with adhesive and covering the vent aperture, wherein the filter comprises:
an adhesive layer comprising the adhesive;
an adsorbent layer on the adhesive layer; and
a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer.

In another embodiment there is provided an ostomy appliance comprising:
a pouch having a vent aperture; and
a filter secured to the pouch with adhesive and covering the vent aperture, wherein the filter comprises:
an adhesive layer comprising the adhesive;
an adsorbent layer on the adhesive layer; and
a gas permeable cover layer secured to the adhesive layer such that the adsorbent layer is covered by the gas permeable cover layer,
wherein the adsorbent layer is positioned on a first side of the adhesive layer so that a first portion of the first side of the adhesive layer is not covered by the adsorbent layer and the gas permeable cover layer is secured to the first portion of the first side of the adhesive layer.

According to a first aspect of the present invention, there is provided an ostomy appliance comprising:
a pouch having a vent aperture; and
a filter secured to the pouch with adhesive and covering the vent aperture, wherein the filter comprises:
   an adhesive layer comprising the adhesive;
   an adsorbent layer on the adhesive layer; and
   a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer,
wherein the adsorbent layer is positioned on a first side of the adhesive layer so that a first portion of the first side of the adhesive layer is not covered by the adsorbent layer and the gas permeable cover layer is secured to the first portion of the first side of the adhesive layer.

As will be appreciated by those skilled in the art; the expression “in register with” means that the outer profiles concerned have the same shape, size, position and alignment. As such, “wherein the gas permeable cover layer has an outer profile and the adhesive layer has a corresponding outer profile which is in register with the outer profile of the gas permeable cover layer” means that the gas permeable cover layer has the same outer profile as the adhesive layer and the outer profile of the gas permeable cover layer is in a state of total alignment with the outer profile of the adhesive layer. In other words, the adhesive layer extends all the way to the outer periphery of the gas permeable cover layer, around the entirety of the periphery of the gas permeable cover layer, but does not extend beyond the outer periphery of the gas permeable cover layer.

In the present invention, the filter is preferably attached to the ostomy bag exclusively with adhesive, meaning the outer profiles of the gas permeable cover layer and adhesive layer can be in register, reducing the footprint of the filter as compared to the filter of EP 2 274 068 B1, which has a flange arranged outward of the adhesive for welding to the ostomy pouch. This lack of a flange has the effect of reducing waste, both in terms of the material of the flange, and in terms of the release liner on which the filter will be supplied, which of course can be made smaller, because the footprint of the filter is reduced. Furthermore, the specific construction of the filter allows the filter to be constructed in a very efficient manner with a minimally sized adsorbent layer, thereby saving material and expense.

The pouch may comprise a wall defining a cavity. The wall may comprise film. The wall may be formed by first and second panels. The wall may be an inner wall.

The pouch may comprise an outer skin. The outer skin may be formed by third and fourth panels.

The outer skin and inner wall may be bonded together, and the filter may be secured to the pouch in a region where the outer skin and inner wall are bonded. The bonding of the outer skin and inner wall together may be accomplished, for example, by welding or the use of adhesive.

The outer skin and inner wall may be welded together, and the filter may be secured to the pouch in a region where the outer skin and inner wall are welded. Accordingly, in one embodiment, there is provided an ostomy appliance comprising:
a pouch having a vent aperture; and
a filter secured to the pouch with adhesive and covering the vent aperture, wherein the filter comprises:
an adhesive layer comprising the adhesive;
an adsorbent layer on the adhesive layer; and
a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer,
wherein the adsorbent layer is positioned on a first side of the adhesive layer so that a first portion of the first side of the adhesive layer is not covered by the adsorbent layer and the gas permeable cover layer is secured to the first portion of the first side of the adhesive layer,
wherein the pouch comprises a cavity, an inner wall defining the cavity and an outer skin, wherein the inner wall and outer skin are welded together, and the filter is secured to the pouch in a region where the inner wall and outer skin are welded.

It is thought that the weld in the region where the filter is attached to the ostomy pouch may improve adhesion of the filter to the pouch.

The outer skin and inner wall may be smash welded together, and the filter may be secured to the pouch in a region where the outer skin and inner wall are smash welded. Accordingly, in one embodiment, there is provided an ostomy appliance comprising:
a pouch having a vent aperture; and
a filter secured to the pouch with adhesive and covering the vent aperture, wherein the filter comprises:
an adhesive layer comprising the adhesive;
an adsorbent layer on the adhesive layer; and
a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer,
wherein the adsorbent layer is positioned on a first side of the adhesive layer so that a first portion of the first side of the adhesive layer is not covered by the adsorbent layer and the gas permeable cover layer is secured to the first portion of the first side of the adhesive layer,
wherein the pouch comprises a cavity, an inner wall defining the cavity and an outer skin, wherein the inner wall and outer skin are welded together, and the filter is secured to the pouch in a region where the inner wall and outer skin are welded,
wherein the inner wall and outer skin are smash welded together, and the filter is secured to the pouch in a region where the inner wall and outer skin are smash welded.

The outer skin and inner wall may comprise the vent aperture.

The pouch may comprise front and back sections which are joined around an outer periphery of the pouch. The front section may comprise the vent aperture and the filter may be provided on the front section. The filter may be spaced from the outer periphery of the pouch. Accordingly, in one embodiment, there is provided an ostomy appliance comprising:
a pouch having a vent aperture; and
a filter secured to the pouch with adhesive and covering the vent aperture, wherein the filter comprises:
an adhesive layer comprising the adhesive;
an adsorbent layer on the adhesive layer; and
a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer,
wherein the adsorbent layer is positioned on a first side of the adhesive layer so that a first portion of the first side of the adhesive layer is not covered by the adsorbent layer and the gas permeable cover layer is secured to the first portion of the first side of the adhesive layer,
wherein the pouch comprises front and back sections which are joined around an outer periphery of the pouch, wherein the front section comprises the vent aperture and the filter is provided on the front section, wherein the filter is spaced from the outer periphery of the pouch.

“Spaced from the outer periphery of the pouch” means that the filter does not touch the outer periphery of the pouch.

The filter may be spaced from the outer periphery of the pouch by a distance that is greater than or equal to a maximum length of the adsorbent layer. “The maximum length of the adsorbent layer” is the greatest extent of the adsorbent layer along its greatest dimension.

The adhesive layer of the filter may be spaced from the outer periphery of the pouch. The adhesive layer may be spaced from the outer periphery of the pouch by a distance that is greater than or equal to a maximum length of the adsorbent layer. The outer skin may comprise a comfort material. The comfort material may be a non-woven material. Alternatively, the comfort material may be a woven material. For example, the comfort material may be a woven or non-woven polyester layer; or a woven cotton, polyester, or “poly-cotton” layer. The outer skin may be the outermost layer of the pouch. The film of the inner wall may be liquid impermeable.

The inner wall may be the innermost layer of the pouch. The inner wall may comprise film. The film may be a plastics material. The film may be a barrier film. The film may comprise PVDC (polyvinylidene dichloride) and/or EVA (ethyl vinyl acetate). For example, the film may be a foil of PVDC and EVA. The film may have a thickness of at least 40 μm, 50 μm, 60 μm, 70 μm, or 75 μm. The film may have a thickness of no more than 100 μm, 90 μm, 80 μm, or 75 μm. In a preferred embodiment the thickness of the film is between 70 μm and 80 μm, for example 75 μm. The film may be of a multi-layer construction. The film may comprise one or more EVA layers. The film may comprise one or more PVDC layers. The film may comprise a first EVA layer, a PVDC layer and a second EVA layer. The PVDC layer may be sandwiched between the first EVA layer and the second EVA layer. The filter may be secured to the first EVA layer.

The filter may be secured to the inner wall. The filter may be secured to the outer skin.

The pouch may have an inlet aperture. A wafer or “base-plate” may be provided around the inlet aperture (i.e. the ostomy appliance may be a “one-piece” appliance). Alternatively, a coupling may be provided around the inlet aperture for coupling to a separate wafer or base plate (i.e. the ostomy appliance may be a “two-piece” appliance).

The pouch may have a drain aperture (i.e. the ostomy appliance may be an “open” appliance). Alternatively the pouch may be a “closed” appliance (with no drain aperture). The inner wall may define the drain aperture.

The adhesive of the adhesive layer which secures the filter to the pouch may have an adhesive layer outer profile that is in register with the outer profile of the gas permeable layer. Accordingly, in one embodiment, there is provided an ostomy appliance comprising:
a pouch having a vent aperture; and
a filter secured to the pouch with adhesive and covering the vent aperture, wherein the filter comprises:
an adhesive layer comprising the adhesive;
an adsorbent layer on the adhesive layer; and
a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer,
wherein the adsorbent layer is positioned on a first side of the adhesive layer so that a first portion of the first side of the adhesive layer is not covered by the adsorbent layer and the gas permeable cover layer is secured to the first portion of the first side of the adhesive layer,
wherein the adhesive of the adhesive layer which secures the filter to the pouch has an adhesive layer outer profile that is in register with the outer profile of the gas permeable layer.

The adhesive layer may comprise an adhesive bearing material and an adhesive. The adhesive may be provided on both sides of the adhesive bearing material.

The adhesive bearing material may be a non-adhesive absorbent material having the adhesive applied thereto.

The adhesive bearing material may have an adhesive bearing material outer profile which corresponds to the outer profile of the gas permeable cover layer and that is in register with the outer profile of the gas permeable cover layer.

A first adhesive layer may be provided on a first adhesive bearing material side of the adhesive bearing material and a second adhesive layer may be provided on a second adhesive bearing material side of the adhesive bearing material. The first adhesive layer may have a first adhesive layer outer profile that corresponds to and is in register with the adhesive bearing material outer profile. The second adhesive layer may have a second adhesive layer outer profile that corresponds to and is in register with the adhesive bearing material outer profile.

The adhesive layer may comprise doubled-sided adhesive tape.

The adhesive layer may comprise one or more materials which are liquid and gas impermeable.

The adhesive layer may comprise pressure sensitive adhesive.

The adhesive layer may comprise a second side. The second side may be opposite the first side.

The second side of the adhesive layer may include an adhesive region.

The adhesive region may be provided across an entirety of the second side of the adhesive layer. Accordingly, in one embodiment, there is provided an ostomy appliance comprising:
a pouch having a vent aperture; and
a filter secured to the pouch with adhesive and covering the vent aperture, wherein the filter comprises:
an adhesive layer comprising the adhesive;
an adsorbent layer on the adhesive layer; and
a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer,
wherein the adsorbent layer is positioned on a first side of the adhesive layer so that a first portion of the first side of the adhesive layer is not covered by the adsorbent layer and the gas permeable cover layer is secured to the first portion of the first side of the adhesive layer,
wherein the adhesive layer comprises a second side that is opposite the first side,
wherein the second side of the adhesive layer includes an adhesive region, wherein the adhesive region is provided across an entirety of the second side of the adhesive layer.

The adhesive layer may comprise an opening. The opening may allow gas access to the adsorbent layer.

The adhesive region may comprise a window which defines the opening. Accordingly, in one embodiment, there is provided an ostomy appliance comprising:
a pouch having a vent aperture; and
a filter secured to the pouch with adhesive and covering the vent aperture, wherein the filter comprises:
an adhesive layer comprising the adhesive;
an adsorbent layer on the adhesive layer; and
a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer,
wherein the adsorbent layer is positioned on a first side of the adhesive layer so that a first portion of the first side of the adhesive layer is not covered by the adsorbent layer and the gas permeable cover layer is secured to the first portion of the first side of the adhesive layer,
wherein the adhesive layer comprises an opening that allows gas access to the adsorbent layer,
wherein the adhesive layer comprises a second side that is opposite the first side,
wherein the second side of the adhesive layer includes an adhesive region,
wherein the adhesive region comprises a window which defines the opening.

The adhesive region may surround the opening.

The adhesive region may overlap an outer perimeter of the adsorbent layer.

The gas permeable cover layer may be liquid impermeable.

The gas permeable cover layer may be microporous.

The gas permeable cover layer may be liquid impermeable.

The gas permeable cover layer may be transparent.

The adsorbent layer may have a non-circular outer profile.

The adsorbent layer may have a substantially rectangular outer profile.

The adsorbent layer may comprise adsorbent particles in a fine fibre web.

The adsorbent layer may comprise activated carbon. The adsorbent layer may be substantially free of binder material.

The adsorbent layer may comprise 1 to 30 weight percent fine fibre having a fibre diameter of 0.001 to 5 micron and 70 to 99 weight percent adsorbent particles.

The adsorbent layer may comprise a support scrim, granular carbon and a polymeric fine-fibre matrix.

The adsorbent layer may comprise a first adsorbent layer side located on the first side of the adhesive layer, a second adsorbent layer side opposite the first adsorbent layer side, and at least one connecting surface connecting the first and second adsorbent layer sides. The gas permeable cover layer may extend around at least one edge where the at least one connecting surface meets the second adsorbent layer side.

The filter may be secured to an inner surface of the pouch. Alternatively, the filter may be secured to an outer surface of the pouch.

The gas permeable cover layer may be directly secured to the adhesive layer.

An intermediate layer may be provided between the adsorbent layer and the adhesive layer.

The adsorbent layer may be directly attached to the adhesive layer.

This is advantageous as the adhesive may serve an additional purpose of attaching the adsorbent layer to the gas permeable cover layer.

The dimensions of the various layers of the filter can vary based on the size of the vent opening and other factors. In some embodiments, the gas permeable cover layer has a width of at least 5 mm, at least 10 mm, or at least 15 mm. Preferably, in an embodiment the gas permeable cover layer has a width of 23 mm, and in another 20 mm. In one embodiment, the width of the gas permeable cover layer is at most 30 mm, and in another, at most 40 mm. In various embodiments, the gas permeable cover layer has a length of at least 10 mm or at least 20 mm. In one embodiment, the gas permeable cover layer has a width of 35 mm, and in another 38 mm. In one embodiment, the width of the gas permeable cover layer is at most 45 mm, and in another, at most 50 mm. The gas permeable cover layer has a thickness of at least 0.5 mils (0.013 mm) in one embodiment, and at most 35 mils (0.9 mm) in another embodiment.

A second portion of the first side of the adhesive layer may be covered by the adsorbent layer. The first portion may be a portion of the first side of the adhesive layer that surrounds the second portion and that extends to a periphery of the adhesive layer. The first portion may have a greater area than the second portion. Alternatively, the second portion may have a greater area than the first portion. Each of the first portion and second portion may be a substantially rectangular ring-shaped area. The first portion may surround the second portion. The second portion may surround the opening.

The invention also provides a method of manufacturing an ostomy appliance comprising adhering, and not welding, a filter to an ostomy pouch. The method may comprise forming a pouch with a vent aperture. The method may comprise securing a filter to the pouch such that the filter covers the vent aperture using adhesive. The filter may comprise an adhesive layer. The filter may comprise an adsorbent layer on the adhesive layer. The filter may comprise a gas permeable cover layer secured to the adhesive layer such that the adsorbent layer is covered by the gas permeable cover layer. The gas permeable cover layer may have an outer profile and the adhesive layer may have a corresponding outer profile which is in register with the outer profile of the gas permeable cover layer. The adsorbent layer may be positioned on a first side of the adhesive layer so that a first portion of the first side of the adhesive layer is not covered by the adsorbent layer. The gas permeable cover layer may be secured to the first portion of the first side of the adhesive layer.

According to a second aspect of the present invention, there is provided a method of manufacturing an ostomy appliance comprising:
forming a pouch with a vent aperture;
securing a filter to the pouch such that the filter covers the vent aperture using adhesive, wherein the filter comprises:
   an adhesive layer;
   an adsorbent layer on the adhesive layer; and
   a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer,
wherein the adsorbent layer is positioned on a first side of the adhesive layer so that a first portion of the first side of the adhesive layer is not covered by the adsorbent layer and the gas permeable cover layer is secured to the first portion of the first side of the adhesive layer,
wherein the securing step does not comprise welding the filter to the pouch.

The method of the second aspect of the present invention may incorporate any or all features of the ostomy appliance of the broad aspect, or the first aspect of the present invention including any optional features.

The pouch may comprise an inner wall and an outer skin and the step of forming a pouch may comprise welding the outer skin and inner wall together. The step of securing the filter to the pouch may comprise securing the filter to the pouch in a region where the inner wall and outer skin are welded.

Welding the outer skin and inner wall together may comprise smash welding the outer skin and inner wall together. The step of securing the filter to the pouch may comprise securing the filter to the pouch in a region where the outer skin is welded to the inner wall.

The outer skin and inner wall may comprise the vent aperture.

The outer skin may be a comfort material and the inner wall may be a film.

The outer skin may be the outermost layer of the pouch when the pouch is formed.

The invention also provides a filter for an ostomy appliance, the filter comprising a gas permeable cover layer having an outer profile and an adhesive layer having a corresponding outer profile which is in register with the outer profile of the gas permeable cover layer. The filter may comprise an adsorbent layer. The adsorbent layer may be on the adhesive layer. The gas permeable cover layer may be secured to the adhesive layer such that the adsorbent layer is covered by the gas permeable cover layer. The adsorbent layer may be positioned on a first side of the adhesive layer so that a first portion of the first side of the adhesive layer is not covered by the adsorbent layer. The gas permeable cover layer may be secured to the first portion of the first side of the adhesive layer.

According to a third aspect of the present invention, there is provided a filter for an ostomy appliance, comprising:
an adhesive layer;
an adsorbent layer on the adhesive layer; and
a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer,
wherein the adsorbent layer is positioned on a first side of the adhesive layer so that a first portion of the first side of the adhesive layer is not covered by the adsorbent layer and the gas permeable cover layer is secured to the first portion of the first side of the adhesive layer.

The filter of the third aspect of the present invention may incorporate any or all features of the filter described in relation to the ostomy appliance of the broad aspect of the invention or the first aspect of the present invention optionally including any optional features.

The adhesive of the adhesive layer may be for attaching the filter to an ostomy pouch.

The filter may further comprise a release layer provided on a second side of the adhesive layer.

A roll of filters may be provided, the filters adhered, via the second side of the adhesive layer, to a roll of release liner. The release liner may have a width corresponding to the width of the filter. Accordingly, because the filter does not require a flange beyond the adhesive for welding to a pouch, and therefore has a smaller footprint, the release liner can be commensurately thinner. This reduces waste, allows for better use of space in factories, and can even allow for use of smaller machines (since the roll of release liner can be smaller).

Moreover, of course, all aspects of the invention can avoid the need for a step of welding the filter to the pouch, thereby avoiding a time-consuming manufacturing step, the heat required to carry out the weld, and the cooling systems necessary to attend to that heat.

### DETAILED DESCRIPTION OF THE INVENTION

In order that the invention may be more clearly understood one or more embodiments thereof will now be described, by way of example only, with reference to the accompanying drawings, of which:

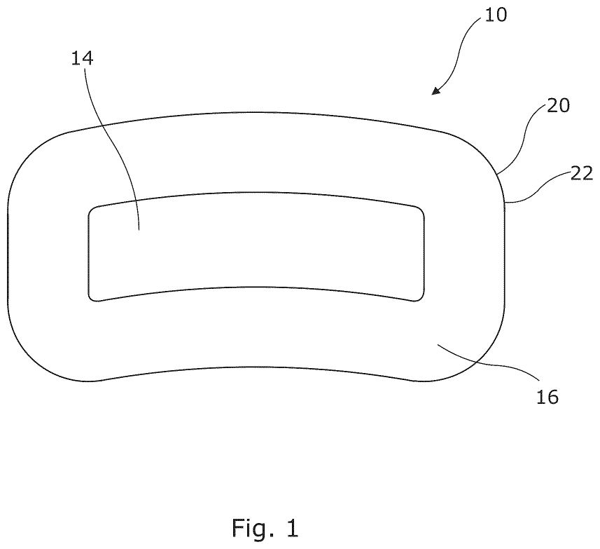

FIG. 1 shows a filter according to the present invention in plan;

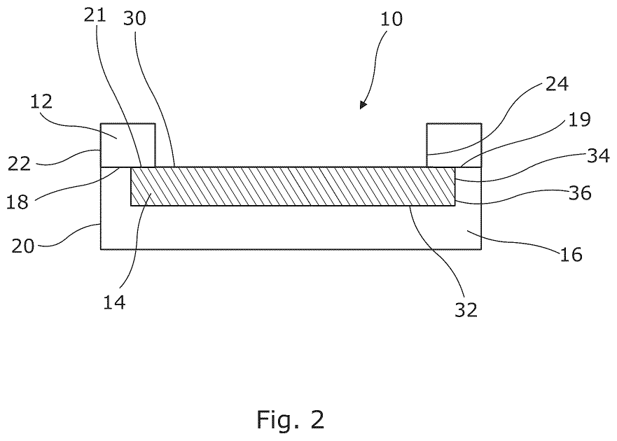

FIG. 2 is a schematic representation showing the filter of FIG. 1 in section;

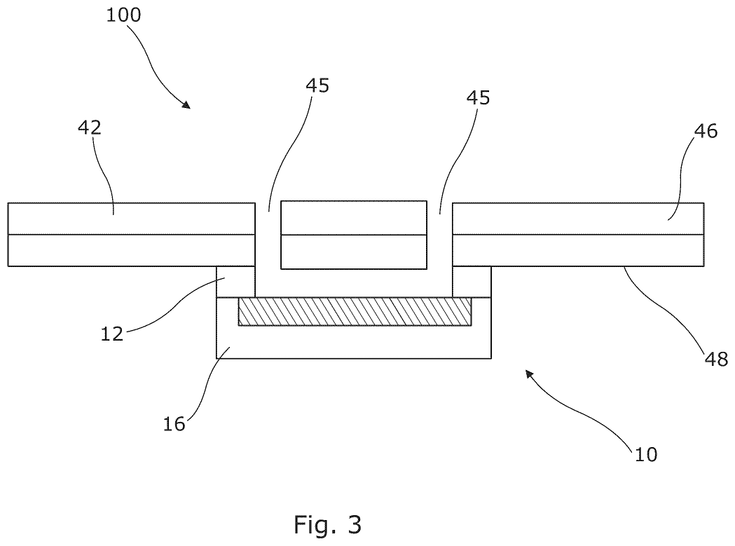

FIG. 3 is a schematic representation showing a portion of a first embodiment of an ostomy appliance incorporating the filter of FIG. 1;

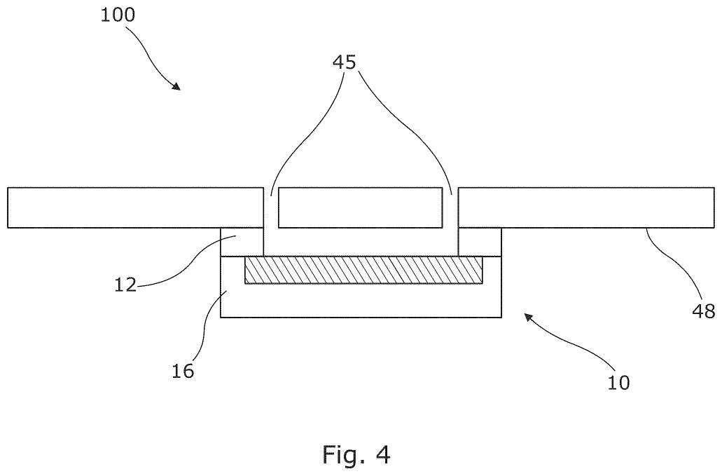

FIG. 4 is a schematic representation showing a portion of a second embodiment of an ostomy appliance incorporating the filter of FIG. 1;

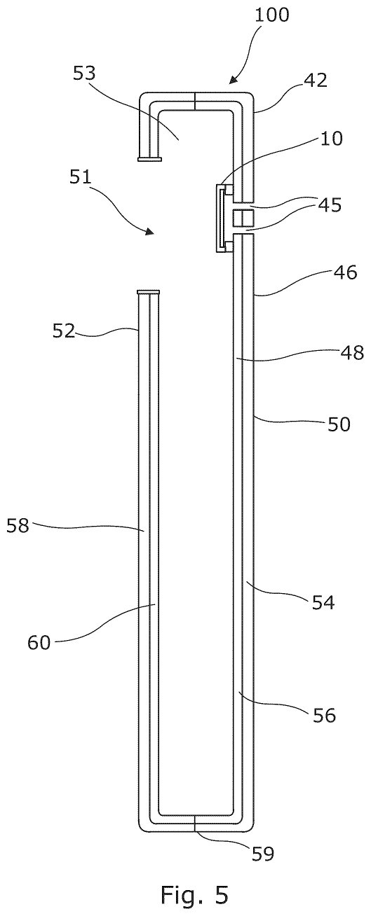

FIG. 5 is a schematic cross-sectional representation showing the ostomy appliance of FIG. 3 in section; and

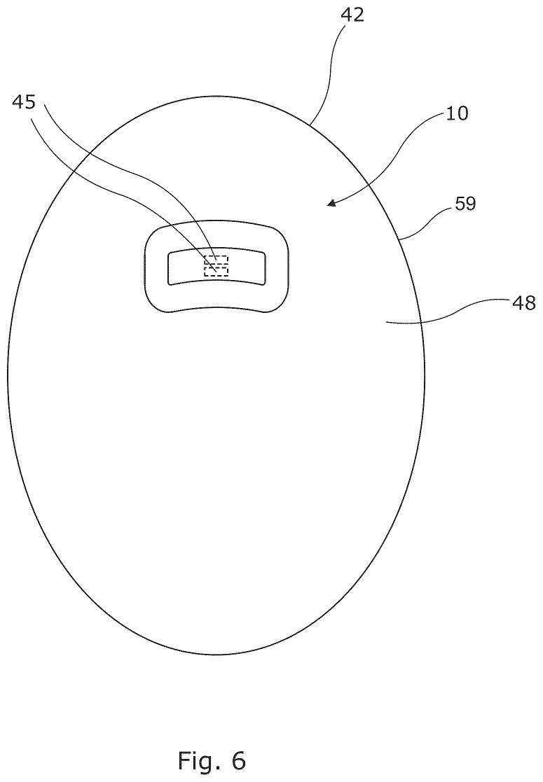

FIG. 6 is a schematic interior plan-view showing the filter of FIG. 1 secured within the ostomy appliance of FIG. 3.

With reference to FIGS. 1 and 2, there is shown a filter 10. As shown the filter includes an adhesive layer 12, adsorbent layer 14 and a gas permeable cover layer 16. However, additional layers may be provided.

The adhesive layer 12 comprises adhesive for securing the filter 10 to a pouch of an ostomy appliance. The adhesive layer 12 may comprise an adhesive bearing material provided with adhesive on each side of the bearing material or alternatively may exclusively comprise adhesive. The adhesive layer may be double-sided adhesive tape. Suitable tapes include 3M 9495 LE Double-Sided Tape sold by 3M United Kingdom PLC of Bracknell, UK. The adhesive is optionally liquid and/or gas impermeable and therefore, in this embodiment, a central opening 24 allowing access to the adsorbent layer 14 is provided in the adhesive layer 12.

The adsorbent layer 14 is positioned on the adhesive layer 12 and the gas permeable cover layer 16 is secured to the adhesive layer 12 such that the gas permeable cover layer 16 covers the adsorbent layer 14. In this embodiment, the gas permeable cover layer 16 is directly secured to the adhesive layer, although one or more intermediate layers may be provided. As shown, the adsorbent layer 14 is positioned on the adhesive layer 12 such that a first portion 18 of a first side 19 of the adhesive layer is left uncovered by the adsorbent layer 14. The gas permeable cover layer 16 is larger than the adsorbent layer, and the first portion 18 which is uncovered by the adsorbent layer 14 is arranged outward of the adsorbent layer, on the cover layer 16. In the depicted example, a second portion 21 of the first side of the adhesive layer is covered by the adsorbent layer 14. Thus, the first portion 18 surrounds the second portion 21 and extends to an outer periphery of the adhesive layer 12. In the depicted example, the second portion 21 extends from the first portion 18 to the opening 24. Each of the first portion 18 and second portion 21 is a substantially rectangular ring-shaped area, and the first portion 18 surrounds the second portion 21. The second portion 21 surrounds the opening 24. The first portion 18 may have a first portion width (i.e. the distance between the second portion 21 and the outer periphery of the adhesive layer) of around 5 mm and the second portion 21 may have a second portion width (i.e. the distance between the first portion and the opening) of around 2.5 mm to 4 mm.

The adsorbent layer 14 of this embodiment comprises adsorbent particles in a fine fibre web, which in this embodiment comprises activated carbon; of course, alternative adsorbent layers could be used. As best shown in FIG. 1, the adsorbent layer 14 in this embodiment has a substantially rectangular outer profile. In particular, in the depicted example, the outer profile of the adsorbent layer 14 is a rectangle with rounded corners. Of course, those skilled in the art will appreciate that other shapes could be used instead.

As shown a second side of the adhesive layer 12 may include an adhesive region that surrounds the opening 24, and the adhesive region may overlap an outer perimeter of the adsorbent layer 14. In particular, the adhesive region of the second side of the adhesive layer 12 is provided across an entirety of the second side of the adhesive layer 12 and comprises a window which defines the opening 24.

As shown, the gas permeable cover layer 16 is secured to the first portion 18 of the adhesive layer 12. In this embodiment, the gas permeable cover layer 16 is directly secured to the first portion 18 of the adhesive layer 12. However, the gas permeable cover layer 16 may be indirectly secured to the first portion 18 of the adhesive layer 12.

In this embodiment, the adsorbent layer 14 comprises a first adsorbent layer side 30, a second adsorbent layer side 32 opposite the first adsorbent layer side 30, and at least one connecting surface 34 connecting the first and second adsorbent layer sides 30, 32. The gas permeable cover layer 16 extends around an edge 36 where the at least one connecting surface 34 meets the second adsorbent layer side 32.

As shown best in FIGS. 1 and 2, the gas permeable cover layer 16 has an outer profile 20 and the adhesive layer 12 has a corresponding outer profile 22 which is in register with the outer profile of the gas permeable cover layer 16. In other words, the gas permeable cover and adhesive layers 16, 12 have the same outer profiles and those profiles are in total alignment. There is no overlap/underlap between the gas permeable cover layer 16 and adhesive layer 12. It should be noted that the adhesive of the adhesive layer 12 which in use secures the filter 10 to the ostomy pouch has an adhesive layer outer profile that is in register with (i.e. the same as) the gas permeable layer 16 of the filter 10.

The gas permeable cover layer 16 can be microporous and/or formed from a transparent material. The gas permeable cover layer 16 may be formed from a polypropylene support scrim, and an ePTFE membrane, which are attached to each other with polyurethane adhesive.

With reference to FIGS. 3 and 5, an ostomy appliance 100 according to the present invention includes the filter 10 of FIG. 2 secured to a pouch 42. The filter 10 is secured to the pouch 42 exclusively with the adhesive of the adhesive layer 12, and such that the filter covers a vent aperture 45 provided in the pouch 42. In the depicted example, there are two vent apertures 45 provided in the pouch and the filter 10 covers both of them. These vent apertures 45 are slits cut into the pouch. In the depicted example, the filter 10 is secured to an inner surface of the pouch 42. However, it should be understood that the filter 10 may instead be secured to an outer surface of the pouch 42.

As shown the pouch 42 may comprise an outer skin 46 and an inner wall 48. In the depicted example, the outer skin 46 is the outermost layer. The inner wall 48 is the innermost layer and defines the pouch cavity 53 into which waste from the user may fall via an inlet aperture 51 provided in the pouch 42. The inner wall 48 comprises a film.

The outer skin 46 is an optional partial or total covering for the inner wall 48 which comprises a comfort material and. In particular, the comfort material used to form the outer skin 46 is a non-woven material, which obscures the content of the bag and is typical of ostomy appliances intended for use outside a hospital environment (In a hospital environment, ostomy appliances are typically provided without a comfort material, in order that health care professionals can easily inspect the contents of the bag through the inner wall 48, which will typically be formed of a transparent film material).

As shown vent apertures 45 provided in the pouch 42 extend completely through the outer skin and inner wall 46, 48. As shown, the inlet aperture 51 is provided in the outer skin 46 and inner wall 48 opposite the vent apertures 45.

FIG. 4 shows an ostomy appliance according to a second embodiment of the present invention. The second embodiment is the same as the first embodiment aside from the fact that the ostomy appliance of the second embodiment does not include a comfort layer. The reference numerals are unchanged with respect to FIG. 3.

With reference to FIG. 5, when the ostomy appliance 100 of the first embodiment is constructed, the first step of the process is to form front and back sections 50 and 52 of the pouch 42. The front section 50 will form the clothing-facing surface of the appliance, on which the filter 10 is provided, and the back section 52 will form the body-facing surface of the appliance, on which a wafer, base-plate or coupling for a wafer or base-plate will be provided.

The front section 50 is formed by welding together a first panel 54 and a second panel 56. The first panel 54 is formed of an outer skin material that will make up the outer skin once the pouch 42 is constructed and the second panel 56 is formed of an inner wall material that will make up the inner wall 48 once the pouch 42 is constructed.

The back section 52 is formed by welding together a third panel 58 of the outer skin material and a fourth panel 60 of the inner wall material. Inlet aperture holes for forming the inlet aperture 51 are cut into the third and fourth panels 58, 60 of the back section 52 before they are welded together to form the inlet aperture. The third and fourth panels 58, 60 are welded in the region of these holes during the welding of the third and fourth panels and a wafer (not shown) or means for coupling a wafer is then fixed in position on the back section such that a patient's stoma may be put into fluid communication with the inlet aperture 51 in use.

When the first and second panels 54, 56 are welded together to form the front section 50 the first and second panels 54, 56 are welded together in the region where the filter is to be applied. Once the first and second panels 54, 56 have been welded together the vent apertures are 45 are cut in the front section 50 using a laser cutting device. During this step the first and second panels 54, 56 are held in alignment by the weld in the region where the filter is to be applied. This ensures that cuts in the first and second panels 54, 56 which form the vent apertures are in register. The filter 10 is then secured to the first panel 54 such that it covers the vent apertures 45 using exclusively adhesive. And

The front section 50 is then secured to the back section 52 by welding the two sections together around their peripheries such that the second and fourth panels 56, 60 form the inner wall 48 defining the cavity 53 and the first and third panels 54, 58 form the outer skin 46 covering the inner wall 48. In particular, the front and back sections 50, 52 are joined together around an outer periphery 59 of the pouch 42.

In this embodiment, smash welding is employed to attach the panels and front and back sections together. However, other types of welding may be employed. Indeed, other methods to welding may be employed to bond the panels, and front and back sections together, such as, for example, using adhesive.

The filter 10 is shown secured to the pouch 42 and covering the vent apertures 45 in FIG. 6. As shown, in this embodiment, the filter 10 is spaced from the outer periphery 59 of the pouch 42.

Adhesive exclusively is used to secure the filter to the pouch and no portion of the filter is welded to the pouch. This removes the need for the filter to include a peripheral flange for welding the filter to the pouch, reducing the footprint of the filter, thereby saving material and reducing waste.

Energy is saved during the manufacture of the ostomy appliance as no welding step is required to attach the filter to the pouch.

The specific construction of the filter allows the filter to be constructed in a very efficient manner with a minimally sized adsorbent layer, thereby saving material and expense.

It is thought that welding the outer skin and inner wall together may help with adhesion of the filter to the pouch.

The one or more embodiments are described above by way of example only. Many variations are possible without departing from the scope of protection afforded by the appended claims.

1. An ostomy appliance comprising:
a pouch having a vent aperture; and
a filter secured to the pouch with adhesive and covering the vent aperture, wherein the filter comprises:
an adhesive layer comprising the adhesive;
an adsorbent layer on the adhesive layer; and
a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer,
wherein the adsorbent layer is positioned on a first side of the adhesive layer so that a first portion of the first side of the adhesive layer is not covered by the adsorbent layer and the gas permeable cover layer is secured to the first portion of the first side of the adhesive layer.

2. The appliance of claim 1, wherein the pouch comprises a cavity, an inner wall defining the cavity and an outer skin, wherein the inner wall and outer skin are welded together, and the filter is secured to the pouch in a region where the inner wall and outer skin are welded.

3. The appliance of claim 2, wherein the inner wall and outer skin are smash welded together, and the filter is secured to the pouch in a region where the inner wall and outer skin are smash welded.

4. The appliance of claim 2 or 3, wherein the inner wall and outer skin comprise the vent aperture.

5. The appliance of any of claims 2 to 4, wherein the outer skin comprises a comfort material and the inner wall comprises a film, and the outer skin is the outermost layer of the pouch.

6. The appliance of any preceding claim, wherein the adsorbent layer has a substantially rectangular outer profile.

7. The appliance of any preceding claim, wherein the adsorbent layer comprises adsorbent particles in a fine fibre web.

8. The appliance of any preceding claim, wherein the gas permeable cover layer is not welded to the pouch.

9. The appliance of any preceding claim, wherein the gas permeable cover layer does not overlap the adhesive layer.

10. The appliance of any preceding claim, wherein the adsorbent layer comprises adsorbent particles in a fine fibre web.

11. The appliance of any preceding claim, wherein the adsorbent layer comprises a first adsorbent layer side located on the first side of the adhesive layer, a second adsorbent layer side opposite the first adsorbent layer side, and at least one connecting surface connecting the first and second adsorbent layer sides, wherein the gas permeable cover layer extends around an edge of the adsorbent layer where the at least one connecting surface meets the second adsorbent layer side.

12. The appliance of any preceding claim, wherein the adhesive is pressure-sensitive adhesive.

13. The appliance of any preceding claim, wherein the filter is secured to an inner surface of the pouch, or the filter is secured to an outer surface of the pouch.

14. The appliance of any preceding claim, wherein the gas permeable cover layer is directly secured to the adhesive layer.

15. The appliance of any preceding claim, wherein the adhesive of the adhesive layer which secures the filter to the pouch has an adhesive layer outer profile that is in register with the outer profile of the gas permeable layer.

16. The appliance of any preceding claim, wherein the adhesive layer comprises a second side that is opposite the first side, wherein the second side of the adhesive layer includes an adhesive region.

17. The appliance of claim 16, wherein the adhesive region is provided across an entirety of the second side of the adhesive layer.

18. The appliance of claim 16 or 17, wherein the adhesive layer comprises an opening that allows gas access to the adsorbent layer, wherein the adhesive region comprises a window which defines the opening.

19. The appliance of any preceding claim, wherein the pouch comprises front and back sections which are joined around an outer periphery of the pouch, wherein the front section comprises the vent aperture and the filter is provided on the front section, wherein the filter is spaced from the outer periphery.

20. A method of manufacturing an ostomy appliance comprising:
forming a pouch with a vent aperture;
securing a filter to the pouch such that the filter covers the vent aperture using adhesive, wherein the filter comprises:
an adhesive layer;
an adsorbent layer on the adhesive layer; and
a gas permeable cover layer secured to the adhesive layer such that the adsorbent layer is covered by the gas permeable cover layer,
wherein the gas permeable cover layer has an outer profile and the adhesive layer has a corresponding outer profile which is in register with the outer profile of the gas permeable cover layer, wherein the adsorbent layer is positioned on a first side of the adhesive layer so that a first portion of the first side of the adhesive layer is not covered by the adsorbent layer and the gas permeable cover layer is secured to the first portion of the first side of the adhesive layer,
wherein the securing step does not comprise welding the filter to the pouch.

21. The method of claim 20, wherein the pouch comprises an inner wall and an outer skin, wherein the forming a pouch step comprises welding the inner wall and outer skin together, wherein the securing step comprises securing the filter to the pouch in a region where the inner wall and outer skin are welded.

22. The method of claim 21, wherein welding the inner wall and outer skin together comprises smash welding the inner wall and outer skin together, wherein the securing step comprises securing the filter to the pouch in a region where the inner wall and outer skin are welded.

23. The method of claim 21 or 22, wherein the inner wall and outer skin comprise the vent aperture.

24. The method of any of claims 21 to 23, wherein the outer skin comprises a comfort material and the inner wall comprises a film.

25. The method of any of claims 21 to 24, wherein the outer skin is the outermost layer of the pouch when the pouch is formed.
